(19) United States
(12) Patent Application Publication
(10) Pub. No.: US 2010/0286021 A1
(43) Pub. Date: Nov. 11, 2010

(54) Methods of Modulating Prokineticin 2 for Treatment of Stress Response and Anxiety-Related Disorders

(76) Inventors: Qun-Yong Zhou, Irvine, CA (US); Jia-Da Li, Irvine, CA (US)

Correspondence Address: DLA PIPER LLP (US), 4365 EXECUTIVE DRIVE, SUITE 1100, SAN DIEGO, CA, 92121-2133, US

(21) Appl. No.: 12/679,471

(22) PCT Filed: Sep. 25, 2008
(86) PCT No.: PCT/US08/77667

§ 371 Date: Jul. 2, 2010

Related U.S. Application Data
(60) Provisional application No. 60/975,110, filed on Sep. 25, 2007.

(51) Int. Cl. C07K 14/00; G01N 33/50; A61P 25/22

(52) U.S. Cl. 514/1.1; 436/501

(57) ABSTRACT

The invention is based on the discovery that an effective therapeutic strategy for ameliorating the symptoms of anxiety-related disorders can be achieved by decreasing levels of PK2 and administering an effective amount of PK2 receptor antagonist. A method of modulating the behavioral response of a subject displaying symptoms of stress responses and/or anxiety-related disorders is disclosed. The disclosed methods indicate that PK2 is an essential regulator of behavioral stress response independent of HPA.

### BACKGROUND OF THE INVENTION

1. Field of the Invention

The invention relates generally to the field of G-protein coupled receptor signaling and therapy and, more specifically to compounds and methods for modulating prokineticin receptor signaling.

2. Background Information

Evidence from multiple disciplinary studies has converged to convincingly demonstrate the pathophysiological importance of stress response for mood disorders such as anxiety and depression. Stress promotes adaptation, but prolonged period of stress or an inability to cope with the stress ultimately leads to wear-and-tear on the body. When subjected to physically or psychologically stressful stimuli, organisms engage in behaviors such as elevated vigilance level, increased anxiety-like behaviors and suppression of feeding and pain perception. Depending on the stressors, stress also evokes other responses such as activation of neuroendocrine and sympathetic nervous system. Stress responses are thought to be initiated and regulated by a biochemical and electrophysiological network in the central nervous system.

The neural circuit containing corticotropin-releasing factor (CRF) has been identified as an important regulator of the stress response. Activation of the hypothalamic-pituitary-adrenocortical (HPA) system by CRF is a key pathway of stress response. Limbic CRF circuit also modulates stress response in a HPA-independent manner. Other modulators such as vasopressin have been implicated in the regulation of stress response. Despite these advances, the neurobiological mechanisms responsible for the behavioral stress response are still largely unknown.

Prokineticins are a pair of newly discovered regulatory peptides that activate two closely related G protein-coupled receptors. A number of biological functions that can be classified as two general categories of cell excitability and cell motility have been assigned to prokineticins. Particularly, prokineticin 2 (PK2), has been demonstrated as an output signal that regulates diverse circadian rhythms controlled by the suprachiasmatic nucleus (SCN). As the receptor of PK2 (PKR2) is also expressed in brain areas that regulate the stress response, including the amygdale, the lateral septum and the paraventricular nucleus (PVN), it is possible that PK2/PKR2 system also participates in the stress response.

### SUMMARY OF THE INVENTION

The present invention illustrates the role of PK2 in stress response. Various acute stressors dramatically up-regulated the expression of PK2 in the PVN, a central nucleus for stress response. Intracerebroventricular (ICV) infusion of PK2 was shown to facilitate the behavioral stress responses, including reduced food intake and increased arousal, anxiety and depression-like behaviors. In contrast, mice lacking the PK2 gene (PK2^{-/-} mice) showed significantly reduced behavioral stress response, as reflected by the reduced anxiety-like, depression-like behaviors, stress-induced analgesia (SIA) and fasting-induced arousal. Thus, PK2 is an essential regulator of behavioral stress response. The present invention also demonstrates that stress response and circadian timing system are linked together at the molecular level.

In one embodiment, the invention provides a method for identifying a PK2 receptor agonist or antagonist contacting a receptor selected from PK2 receptor and PK1 receptor. The PK receptor may be the PK1 receptor (PKR1) or the PK2 receptor (PKR2) with one or more candidate compounds under conditions wherein PK2 promotes or reduces a predetermined signal. Exemplary predetermined signals include, but are not limited to, calcium ion mobilization.

In another embodiment, the PK2 receptor agonist can be identified by contacting a receptor selected from PK2 receptor and PK1 receptor with one or more candidate compounds under conditions wherein PK2 promotes a predetermined signal and identifying a compound that promotes the predetermined signal, for example, calcium ion mobilization.

In another embodiment, the PK2 receptor antagonist can be identified by contacting a receptor selected from PK2 receptor and PK1 receptor with one or more candidate compounds in the presence of a receptor agonist under conditions wherein the agonist binds to the selected receptor and identifying a compound that reduces the binding. The PK2 receptor agonist can be identified by contacting a receptor selected from PK2 receptor and PK1 receptor with one or more candidate compounds in the presence of a receptor antagonist under conditions wherein the antagonist binds to a selected receptor and identifying a compound that binds to and activates the selected receptor.

In another embodiment, the invention provides a method of modulating behavioral stress response or anxiety-like behaviors in a subject by administering an effective amount of PK2 receptor binding agent. Exemplary binding agents, include, but are not limited to, small molecules, polypeptides, polynucleotides encoding polypeptides that bind PK2 receptor, antibodies or any other modulators known in the art, which decrease the behavioral stress response and/or anxiety-like behaviors in subjects presenting symptoms thereof.

### BRIEF DESCRIPTION OF THE DRAWINGS

FIGS. 1A-1C show induction of PK2 mRNA in the PVN after acute stress. (a) Time course of PK2 mRNA in the PVN after 3-min cold-water swimming; n=3-5 mice/time point; *, P<0.05; unpaired t test. Error bars, SEM. (b) Representative in situ hybridization images of PK2 mRNA before (C) or 2 hours after 3-min cold-water swimming (S). The PVN area is boxed. Scale bar, 1 mm. (c) Autoradiographic emulsion images of the in situ hybridized PVN, as the boxed area in (b). The hybridization signals appear as reduced silver grains in white. Scale bar, 100 μm.

FIGS. 2A-2D shows an effect of intracerebroventricular (ICV) infusion of PK2 on the sleep/wakefulness (a), food intake (b), anxiety-like behavior (c) and depression-like behavior (d). (a) Artificial cerebrospinal fluid (CSF) or 5 μg of PK2 was infused ICV to rats at ZT 14. Slow wave sleep (SWS), rapid eye movement sleep (REM) and active wake (AW) during the following 4 hours (ZT 14-18) were averaged and presented as mean±SEM; n=6 rats/group. (b) CSF or 5 μg of PK2 was infused ICV to rats at ZT 2 (day) or ZT 14 (night), the food intake in the following 24 hours were measured periodically and presented as mean±SEM. n=6-10 rats/group. (c) CSF or 4 μg of PK2 was infused ICV into mice. Anxiety-like behavior was monitored with a light-dark box assay 1 hour after infusion. n=12 mice/group. (d) CSF or 4 μg of PK2 was infused ICV into mice. Immobility in a forced swimming test was measured 1 hour after infusion; n=12 mice/group. *, P<0.05; unpaired t test. Error bars, SEM.

FIGS. 3A-3C demonstrate impaired response of PK2^{-/-} mice to new environments. (a) The food intake of WT (closed circles) and PK2^{-/-} (open circles) mice during day 1 and day 3-4 in Comprehensive Laboratory Animal Monitor System (CLAMS) chambers; n=12 mice/genotype. (b) The core body temperature before and following a cage change at ZT 4. (c) The locomotor activity before and following a cage change at ZT4; n=6 mice/genotype. *, P<0.05.***, P<0.005; unpaired t test. Error bars, SEM. The lines under the curve of (b) and (c) indicate the P values between genotypes are less than 0.05. WT (closed circles), PK2^{-/-} (open circles).

FIGS. 4A-4C show reduced anxiety-like and depression-like behaviors in PK2^{-/-} mice. (a) PK2^{-/-} mice showed reduced anxiety-like behaviors in elevated plus maze; n=15 mice/genotype. (b) PK2^{-/-} mice showed reduced anxiety-like behaviors with a light-dark box assay. n=24 mice/genotype. (c) PK2^{-/-} mice showed antidepression-like behavior in a forced swimming test; n=15 mice/genotype. *, P<0.05; ***, P<0.005; unpaired t test. Error bars, SEM.

FIGS. 5A-5D show impaired fasting-induced arousal in PK2^{-/-} mice. (a and c) time course of wakefulness (W) and non-REM sleep (NREM) in wild-type mice (a) or PK2^{-/-} mice (c) during ad lib feeding (circle) or fasting (triangle); n=6 mice/genotype. Solid bars, dark phase; empty bars, light phase. Arrowheads indicate the time at which food was removed from the cages for the fasting portion of the study that followed baseline recording. Dotted lines demarcate periods quantified in (b) and (d). (b and d) Data from 25 wild-type (b) and PK2^{-/-} mice (d) were collapsed into 6 hour bins over the periods indicated in (a) or (c) for comparison of time spent on wakefulness, non REM sleep and REM sleep. Data are presented in mean±SEM. *, P<0.05; **, P<0.01; unpaired t test.

FIGS. 6A-6C show impaired stress-induced analgesia in PK2^{-/-} mice. The pain test was carried out on a hot-plate of 55° C. surface, on which WT and PK2^{-/-} mice did not show a baseline difference. (a and b) The hot-plate latency was monitored before or after stress. Animals were stressed by 3-min of cold-water swimming (a) or 5-min of tail suspension (b). Wild-type (closed circles); PK2^{-/-} (open circles). The black blocks indicate the stressing period. (c) The hot-plate latency was also monitored after subcutaneous injection of various concentrations of morphine. There was no genotypic difference in morphine-induced analgesia; n=12-15 mice/genotype *, P<0.05; unpaired t test. Error bars, SEM.

FIGS. 7A-7B show the HPA activation in WT and PK2^{-/-} mice. (a) The corticosterone levels in WT and PK2^{-/-} mice before and after 3-min of cold-water swimming. Wild-type (closed circles); PK2^{-/-} (open circles); n=8-10 mice/genotype. *, P<0.05; unpaired t test. Error bars, SEM. (b) Representative in situ images of CRF mRNA in the PVN from WT and PK2^{-/-} mice. There was no genetic difference in CRF mRNA level in the PVN. Scale bar, 500 μm.

### DETAILED DESCRIPTION OF THE INVENTION

The present invention provides methods of modulating the behavioral response of a subject displaying symptoms of stress responses and/or anxiety-related disorders. The invention is based on the discovery that an effective therapeutic strategy for ameliorating the symptoms of anxiety-related disorders can be achieved by decreasing levels of PK2 and administering an effective amount of PK2 receptor antagonist. Thus, PK2 is an essential regulator of behavioral stress response independent of HPA.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “modulating” when used in reference to circadian rhythm is intended to mean altering a physiological function, endocrine function or behavior that is regulated by the circadian timing system of an animal, or altering a cellular function that exhibits circadian rhythmicity. Exemplary physiological functions regulated by the circadian timing system of an animal include body temperature, autonomic regulation, metabolism, and sleep-wake cycles. Exemplary metabolic functions include control of weight gain and loss, including increase or decrease in body weight and increase or decrease in percent body fat. Exemplary endocrine functions regulated by the circadian timing system of an animal include pineal melatonin secretion, ACTH-cortisol secretion, thyroid stimulating hormone secretion, growth hormone secretion, neuropeptide Y secretion, serotonin secretion, insulin-like growth factor type I secretion, adrenocorticotropic hormone secretion, prolactin secretion, gamma-aminobutyric acid secretion and catecholamine secretion. Exemplary behaviors regulated by the circadian timing system of an animal include movement (locomotor rhythm), mental alertness, memory, sensorimotor integration, feeding, REM sleep, NREM sleep and emotion. Exemplary cellular functions that exhibit circadian rhythmicity are neuron firing and transcriptional control of gene expression.

As used herein, the terms “sample” and “biological sample” refer to any sample suitable for the methods provided by the present invention. In one embodiment, the biological sample of the present invention is a tissue sample, e.g., a biopsy specimen such as samples from a needle biopsy. In other embodiments, the biological sample of the present invention is a sample of bodily fluid, e.g., serum, plasma, cerebral spinal fluid, sputum, urine, and ejaculate.

“Defined-medium conditions” or equivalents thereof, e.g., “suitable conditions” refer to environments for culturing cells where the concentration of components therein required for optimal growth are detailed. For example, depending on the use of the cells (e.g., therapeutic applications), removing cells from conditions that contain xenogenic proteins is important (for example, the culture conditions are animal-free conditions or free of non-human animal proteins).

The terms “agonism’ and “antagonism” or equivalents thereof are well known in the art. The term “modulatory effect” or equivalents thereof, refers to the ability of the ligand, including an agonist or antagonist, to change the activity through binding to a ligand binding site.

As used herein, the term “agonist” denotes any molecule that binds to a receptor and initiates a change in the function of a cell. Agonists, as used herein, have attraction to bind to a given receptor and will activate the receptor and subsequently lead to a change in the function of the cell.

As used herein, the term “antagonist” denotes any molecule that, when bound to a receptor, block the receptor and prevents it from responding. Antagonists, as used herein, may prevent agonists from binding, or attaching to the receptor.

The term “allosteric modulator” as used herein denotes a compound that can regulate the activity of a prokineticin or GABA receptor. The allosteric modulator can regulate the activity of a prokineticin or GABA receptor in several ways, for example, by increasing the affinity of a prokineticin receptor for its agonists and/or antagonists or decreasing the affinity of a prokineticin or GABA receptor for its agonists or antagonists. It can also regulate the prokineticin or GABA receptor's activity by effecting the association or dissociation of a prokineticin or GABA receptor agonist or antagonist. Compounds that affect the affinity of prokineticin or GABA receptors for their natural ligand are well known in the art.

In one embodiment, the invention provides methods for identifying an agent that modulates behavioural stress response or anxiety-like behaviours in a subject. As such, the ability of a test agent to modulate an effect of prokineticin can be detected using methods as disclosed herein or otherwise known in the art. The term “test agent” or “test molecule” or “test compound” is used broadly herein to mean any agent or compound that is being examined for agonist or antagonist activity in a method of the invention. Although the method generally is used as a screening assay to identify previously unknown molecules that can act as agonist or antagonist agents as described herein, the methods also can be used to confirm that an agent known to have a particular activity in fact has that activity, such as standardizing the activity of the agent.

A variety of in vitro screening methods are useful for identifying a PK2 receptor antagonist or agonist to be provided in the methods of the invention for identifying a compound that modulates circadian rhythm. The ability of a compound to modulate PK2 receptor can be indicated, for example, by the ability of the compound to bind to and activate PK2 receptor, block agonist binding to PK2 receptor, promote a predetermined signal produced by a PK2 receptor, or reduce a predetermined signal produced by a PK2 receptor. Therefore, signaling and binding assays can be used to identify a PK2 receptor antagonist or agonist that is provided by the methods of the invention for identifying a compound that modulates circadian rhythm.

As used herein, the term “predetermined signal” is intended to mean a readout, detectable by any analytical means, that is a qualitative or quantitative indication of activation of G-protein-dependent signal transduction through PK2 receptor. Assays used to determine such qualitative or quantitative activation of G-protein-dependent signal transduction through PK2 receptor, are referred to below as “signaling assays.” G-proteins, or heterotrimeric GTP binding proteins, are signal transducing polypeptides having subunits designated Gα, Gβ and Gγ, that couple to seven-transmembrane cell surface receptors. G-proteins couple to such receptors to transduce a variety of extracellular stimuli, including light, neurotransmitters, hormones and odorants to various intracellular effector proteins. G-proteins are present in both eukaryotic and prokaryotic organisms, including mammals, other vertebrates, flies and yeast.

A signaling or binding assay used to identify a PK2 receptor antagonist or agonist can contain a PK2 receptor or a PK1 receptor. Because of the homology between PK2 and PK1 receptors, which have amino acid sequences that are about 85% identical, a PK2 receptor or PK1 receptor can be used in screening assays to identify a PK2 receptor agonist. Specifically, due to the homology between the PK1 receptor and PK2 receptor, a PK1 receptor agonist or antagonist is likely to also function as a PK2 receptor agonist or antagonist. Similarly, either PK1 or PK2 can function as an agonist in signaling and binding assay formats that employ a competitive agonist.

As used herein, the term “PK2 receptor agonist” is intended to mean a compound that selectively promotes or enhances normal signal transduction through the PK2 receptor. A PK2 receptor agonist can act by any agonistic mechanism, such as by binding a PK2 receptor at the normal PK2 binding site, thereby promoting PK2 receptor signaling. A PK2 receptor agonist can also act, for example, by potentiating the binding activity of PK2 or signaling activity of PK2 receptor. A PK2 receptor agonist can also be a PK1 receptor agonist. As such, a PK1 receptor agonist can be tested for its ability to function as a PK2 receptor agonist using the screening methods described herein.

A PK2 receptor agonist can include a modification of PK2 or PK1 that is capable of binding to and activating a PK2 receptor. Such a modification can be, for example, one or more additions, deletions or substitutions compared with the recited amino acid sequence; one or more chemical or enzymatic modifications to the polypeptide; or substitution of one or more L-configuration amino acids with corresponding D-configuration amino acids.

As used herein, the term “PK2 receptor antagonist” is intended to mean a compound that selectively inhibits or decreases normal signal transduction through the PK2 receptor. A PK2 receptor antagonist can act by any antagonistic mechanism, such as by binding a PK2 receptor or PK2, thereby inhibiting binding between PK2 and PK2 receptor. A PK2 receptor antagonist can also inhibit binding between a specific or non-specific PK2 receptor agonist and PK2 receptor. Such a specific or non-specific PK2 receptor agonist can be, for example, a drug that produces unwanted side effects by promoting signaling through the PK2 receptor. A PK2 receptor antagonist can also act, for example, by inhibiting the binding activity of PK2 or signaling activity of PK2 receptor. For example, a PK2 receptor antagonist can act by altering the state of phosphorylation or glycosylation of PK2 receptor. A PK2 receptor antagonist can also be an inverse agonist, which decreases PK2 receptor signaling from a baseline amount of constitutive PK2 receptor signaling activity.

When a signaling assay is used to identify a PK2 receptor antagonist or agonist, the methods of the invention can involve contacting a PK1 receptor or PK2 receptor with one or more candidate compounds under conditions in which PK2 promotes a predetermined signal and identifying a compound that either decreases or increases the predetermined signal, respectively. When a binding assay is used to identify a PK2 receptor antagonist or agonist, the methods of the invention can involve contacting a PK1 receptor or PK2 receptor with one or more candidate compounds under conditions in which PK2 binds to the PK2 receptor and identifying a compound that either decreases binding of a PK2 receptor agonist to the PK1 receptor or PK2 receptor, or binds to and activates the PK1 receptor or PK2 receptor, respectively.

Further, a large number of published GPCR structure-function studies have indicated regions of GPCRs involved in ligand interaction, G-protein coupling and in forming transmembrane regions, and indicate regions of GPCRs tolerant to modification. In addition, computer programs known in the art can be used to determine which amino acid residues of a GPCR, such as a mouse PK2 receptor, can be modified as described above without abolishing activity.

A signaling assay can be performed to determine whether a candidate compound is a PK2 receptor agonist or antagonist. In such an assay, a PK2 receptor is contacted with one or more candidate compounds under conditions wherein the PK2 receptor produces a predetermined signal in response to a PK2 agonist, such as PK2. In response to PK2 receptor activation, a predetermined signal can increase or a decrease from an unstimulated PK2 receptor baseline signal. In one embodiment, a predetermined signal may be an increasing signal, for example, when the amount of detected second messenger molecule is increased in response to PK2 receptor activation. In another embodiment, a predetermined signal may be a decreasing signal, for example, when the detected second messenger molecule is destroyed by hydrolysis in response to PK2 receptor activation. A predetermined signal in response PK2 receptor activation can therefore be an increase in a predetermined signal that correlates with increased PK2 receptor activity, or a decrease in a predetermined signal that correlates with increased PK2 receptor activity. Accordingly, a PK2 receptor signaling assay can be used to identify a PK2 receptor agonist that promotes production of a predetermined signal, whether the agonist promotes an increase in a predetermined signal that positively correlates with PK2 receptor activity, or a decrease in a predetermined signal that negatively correlates with PK2 receptor activity. Similarly, a signaling assay can be performed to determine whether a candidate compound is a PK2 receptor antagonist. In such a signaling assay, a PK2 receptor is contacted with one or more candidate compounds under conditions wherein, the PK2 receptor produces a predetermined signal in response to a PK2 receptor agonist, such as PK2, and a compound is identified that reduces production of the predetermined signal.

Signaling through G proteins can lead to increased or decreased production or liberation of second messengers, including, for example, arachidonic acid, acetylcholine, diacylglycerol, cGMP, cAMP, inositol phosphate, such as inositol-1,4,5-trisphosphate, and ions, including Ca²⁺ ions; altered cell membrane potential; GTP hydrolysis; influx or efflux of amino acids; increased or decreased phosphorylation of intracellular proteins; or activation of transcription.

Various assays, including high throughput automated screening assays, to identify alterations in G-protein coupled signal transduction pathways are well known in the art. Various screening assays known in the art that measure Ca²⁺, cAMP, voltage changes and gene expression are envisioned for use with the present invention. Yeast cell-based bioassays for high-throughput screening of drug targets for G-protein coupled receptors may also be utilized. A variety of cell-based expression systems, including bacterial, yeast, baculovirus/insect systems and mammalian cells, useful for detecting G-protein coupled receptor agonists and antagonists may also be utilized.

In one embodiment, assays to detect and measure G-protein-coupled signal transduction can involve the preliminary step of contacting a sample containing PK1 receptor or PK2 receptor, such as an isolated cell, membrane or artificial membrane, such as a liposome or micelle, with a detectable indicator. A detectable indicator can be any molecule that exhibits a detectable difference in a physical or chemical property in the presence of the substance being measured, such as a color change. Calcium indicators, pH indicators, and metal ion indicators, and assays for using these indicators to detect and measure selected signal transduction pathways are known in the art and may be utilized in the present invention. For example, calcium indicators and their use are well known in the art, and include compounds such as, but not limited to Fluo-3 AM, Fura-2, Indo-1, FURA RED, CALCIUM GREEN, CALCIUM ORANGE, CALCIUM CRIMSON, BTC, OREGON GREEN BAPTA, which are available from Molecular Probes, Inc., Eugene Oreg., and described, for example, in U.S. Pat. Nos. 5,453,517, 5,501,980 and 4,849,362.

A variety of nucleic acid molecules can be operatively linked to a PK2 gene promoter. As used herein, the term “operatively linked” is intended to mean that the nucleic acid molecule is positioned with respect to a promoter, such as a mouse or human PK2 promoter, in such a manner that the promoter will direct the transcription of RNA using the nucleic acid molecule as a template.

Methods for operatively linking a nucleic acid to a heterologous promoter are well known in the art and include, for example, cloning the nucleic acid into a vector containing the desired promoter, or appending the promoter to a nucleic acid sequence using PCR. A nucleic acid molecule operatively linked to a promoter of RNA transcription can be used to express reporter nucleic acid transcripts and polypeptides in a desired host cell or in vitro transcription-translation system. Exemplary reporter nucleic acids include luciferase, β-lactamase, β-glucuronidase, green fluorescent protein, blue fluorescent protein, chloramphenicol acetyltransferase and β-galactosidase. Compounds for screening can be contained within large libraries of compounds, such as when high-throughput in vitro screening formats are used. Methods for producing large libraries of chemical compounds, including simple or complex organic molecules, metal-containing compounds, carbohydrates, peptides, proteins, peptidomimetics, glycoproteins, lipoproteins, nucleic acids, antibodies, and the like, are well known in the art and suitable for use with the present invention. Libraries containing large numbers of natural and synthetic compounds also can be obtained from commercial sources.

Compounds can be screened individually or in pools of a few, tens or hundreds of compounds. Therefore, a library of compounds can be screened sequentially, in a multi-sample format, in which each sample receives one compound, or multiplexed format, in which each sample receives more than one compound.

For the in vitro screening methods, a PK2 receptor or PK2 promoter can be contained in a cell preparation. As used herein, the term “cell preparation” is intended to mean a sample containing an isolated cell, which can be a cell contained in an organ, tissue, or cell culture and which contains a naturally occurring PK2 receptor. A cell preparation may include intact, broken, solubilized, homogenized, or fractionated cells in the presence of a wide variety of components, such as buffers, salts and detergents, so long as PK2 receptor is capable of binding to PK2 and becoming activated in response to PK2 binding. A cell preparation also can be a cell line that expresses PK2 receptor. A cell line that expresses PK2 receptor may include identified by methods known in the art, such as competitive binding assays. An exemplary cell line that expresses PK2 receptor is the melanoma cell line M2A7 (available from American Type Culture Collection as ATCC CRL-2500). Other cell lines that express PK2 receptor include M2 melanoma cells and RC-4B/C pituitary tumor cells (ATCC CRL-1903). A cell preparation may also include cells that recombinantly express PK2 receptor. A cell preparation can be obtained from a variety of animals, including, for example, humans, non-human primates, rats and mice. A human cell preparation, for example, is a sample containing an isolated human cell, which can be a cell contained in an organ, tissue, or cell culture and which contains a naturally occurring human PK2 receptor.

As used herein, the term “nucleic acid molecule” refers to a polynucleotide, including an oligonucleotide, of natural or synthetic origin, which can be single— or double— can correspond to genomic DNA, cDNA or RNA, and can represent either the sense or antisense strand or both. The term “nucleic acid molecule” is intended to include nucleic acid molecules that contain one or more non-natural nucleotides, such as nucleotides having modifications to the base, the sugar, or the phosphate portion, or having one or more non-natural linkages, such as phosphorothioate linkages. Such modifications can be advantageous in increasing the stability of the nucleic acid molecule, particularly when used in hybridization applications.

As used herein, the term “isolated nucleic acid molecule” is intended to mean that the nucleic acid molecule is altered, by the hand of man, from how it is found in its natural environment. For example, an isolated nucleic acid molecule can be a molecule operatively linked to an exogenous nucleic acid sequence. An isolated nucleic acid molecule can also be a molecule removed from some or all of its normal flanking nucleic acid sequences.

An isolated molecule can alternatively, or additionally, be a “substantially pure” molecule, in that the molecule is at least 60%, 70%, 80%, 90 or 95% free from cellular components with which it is naturally associated. An isolated nucleic acid molecule can be in any form, such as in a buffered solution, a suspension, a lyophilized powder, attached to a solid support (for example, as a component of a DNA array), or in a cell.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLE I

### Acute Physiological Stressors Induced PK2 Expression in the PVN

This example illustrates that under fasting conditions (24 hours) PK2 mRNA is dramatically induced.

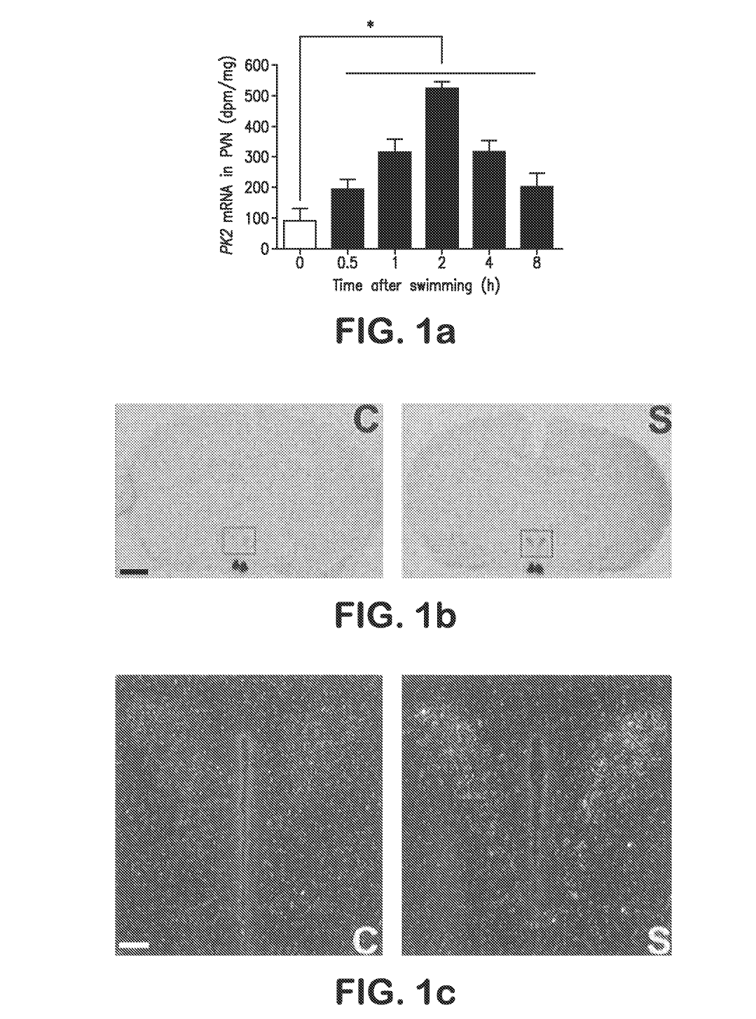

In previous studies dramatic and rapid PK2 up-regulation in the SCN after light stimulation has been demonstrated. As a control experiment, animals were subjected to starvation. Whereas, the PK2 expression in the SCN did not respond to starvation, it was observed that 24 hours of food deprivation dramatically induced PK2 mRNA in the PVN, an area that does not express PK2 under normal feeding conditions. Complete brain map analysis indicated that only the PVN exhibits a change in PK2 expression in responding to starvation. Further studies indicate that the induction was mimicked by 2-Dexoyl-D-glucose (2-DG) administration, whereas PK2 mRNA level fell to the basal level at 4 hours after re-feeding. The central role of the PVN in stress response prompted us to further examine whether the PK2 expression responds to other stressors. As shown in FIG. 1, three minutes of cold-water swimming (10° C.) induced a significant PK2 expression specifically in the PVN. The induction occurred as early as 30 min after swimming, reached peak value at 2 hours, and was still visible at 8 hours after swimming.

As provided herein, the expression of PK2 was dramatically up-regulated by various acute stressors in the PVN, a central nucleus for stress response, where PK2 expression was undetectable under normal conditions. Second, PK2 infusion led to similar behavioral responses elicited by stressors, including suppression of food intake and increase of arousal, anxiety-like and depression-like behaviors. Third, mice deficient in PK2 displayed significantly impaired response to stress; including reduced anxiety-like and depression-like behaviors, as well as impaired stress-induced analgesia and fasting-induced arousal. The intact activation of HPA axis by stress in PK2^{-/-} mice indicates that PK2 modulates behavioral responses under stress is probably independent of HPA system function.

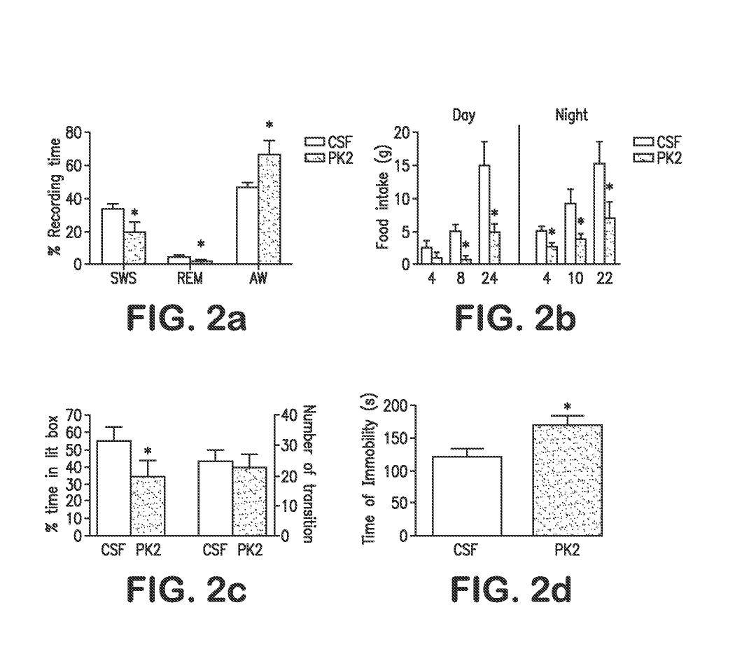

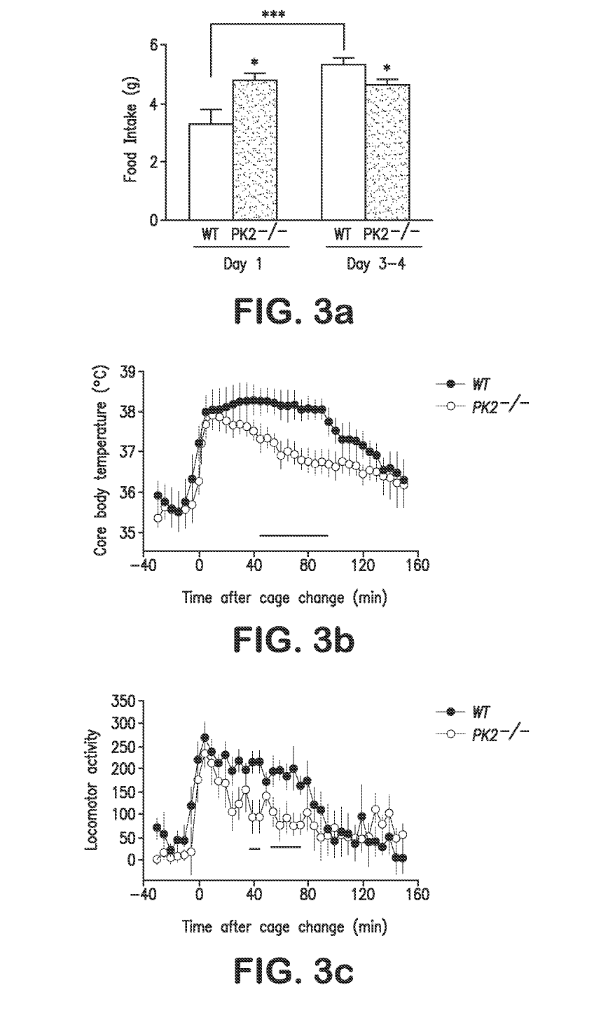

Elevated anxiety level is an important behavioral adaptation when animals encounter a stressor, either physical or psychological. PK2 infusion increased the anxiety-like level (FIG. 2C), while PK2^{-/-} mice showed reduced anxiety-like behaviors (FIG. 3A and 3B), indicating PK2 facilitates the anxiety-like behaviors in mammals. The time scale for these anxiety-like behaviors expanded from minutes (light-dark box and elevated plus maze), to hours (cage change), even days (metabolic cages), consistently indicating the impaired anxiety response in PK2^{-/-} mice. It should be noted that the body temperature and locomotor activity initially reached to the similar peak values after cage change in both wild-type and PK2^{-/-} mice (FIGS. 3B and 3C), indicating the intact pathways of stress perception in PK2^{-/-} mice.

The forced swimming test is conducted by exposing animals to an inescapable and uncontrollable threatening situation. Since many antidepressants reduce immobility in the forced swimming test, an increase in immobility has been interpreted as the development of behavioral despair. However, an alterative interpretation of forced swimming is that alternating periods of swimming and immobility reflect a search-and-wait strategy of coping with an uncontrollable stressor. In this case, higher levels of immobility would have a more adaptive value, so that the animals would be able to conserve energy for successive bouts of swimming. Accordingly, the present invention demonstrates that PK2 infusion leads to increased immobility indicates that PK2 signaling facilitates this coping behavior. As such, while reduced levels of immobility displayed by PK2 mice may be considered as having reduced coping ability.

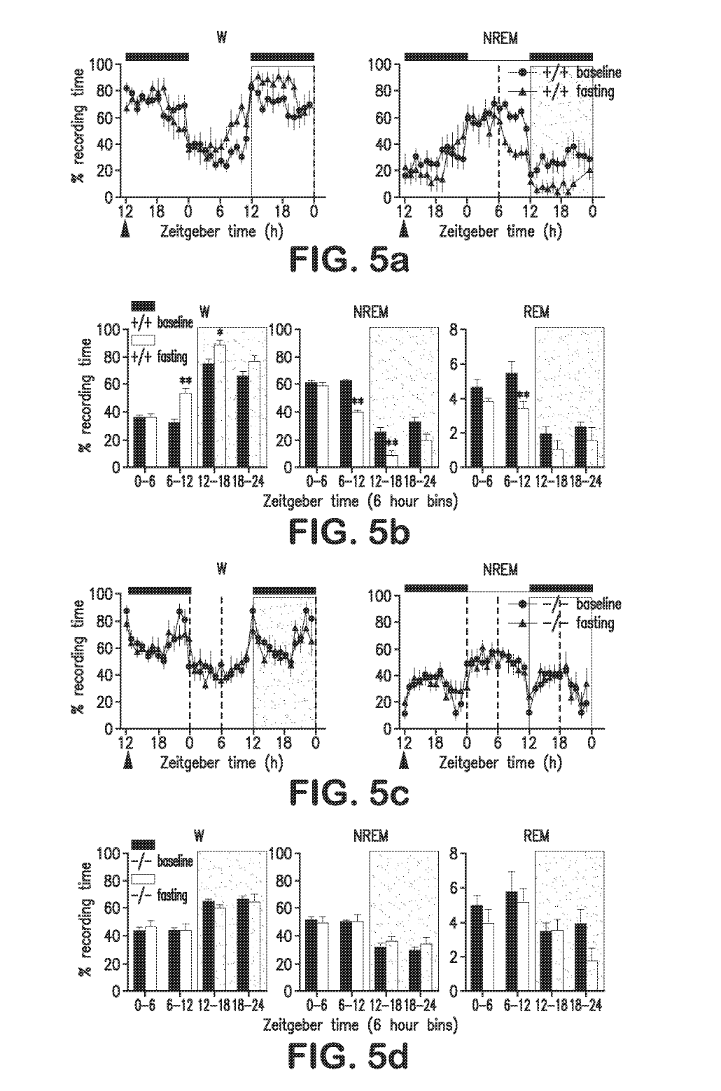

Elevated vigilance during stressful situations is advantageous for animals to cope with stressors. In response to stress of starvation, animals adapt acutely by becoming more wakeful and active, a response that presumably enhance the ability to seek food. Significant increase of wakefulness in wild-type mice was observed after fasting for 18 hours (FIGS. 5A and 5B). In contrast, PK2^{-/-} mice failed to exhibit this fasting-induced arousal and kept their sleep/wake pattern almost identically during fasting and ad lib feeding (FIGS. 5C and 5D). These studies, together with the observation that PK2 infusion enhances arousal as well as starvation dramatically up-regulated PK2 mRNA in the PVN, indicate that PK2 signaling is critically involved in stress- and starvation-induced arousal response.

The perception of pain detects harmful mechanical, thermal, or chemical stimuli and is therefore physiologically advantageous. However, pain perception can be temporarily masked by a variety of stressors, termed as SIA (Stress-induced antinociception), as part of stress responses. Similar to other impaired behavioral responses to stress, SIA was greatly attenuated in PK2^{-/-} mice. PVN, through its afferent and efferent nervous fibers, has the reciprocal connection with the other brain nuclei such as periaqueductal gray (PAG), raphes magnus nucleus and raphes dorsalis nucleus, and spinal cord regions including dorsal horn, which contain neural structures involved in the nociception. There exists ample evidences that support the critical role of PVN in antinociception; for instance, direct stimulation of PVN enhanced the analgesia, whereas PVN cauterization weakened the analgesia. Stress, such as cold-water swimming, induced PK2 expression in the PVN; moreover, the PK2 receptor (PKR2) is highly expressed in the PVN, implying the PVN might be an important brain area for PK2 to execute the SIA. It should be noted that PK2 is also expressed in amygdale and PKR2 is expressed in the dorsomedial PAG, thus the amygdale/PAG pathway may be a possible alternative SIA route for PK2.

Three minutes of cold-water swimming led to a striking increase of the corticosterone level in both wild-type and PK2^{-/-} mice. In fact, PK2^{-/-} mice displayed even greater increase of corticosterone level in response to cold-water swimming. This result indicates that the stress can be perceived by PK2^{-/-} mice and it elicited the activation of HPA axis in these mice. The present invention demonstrates that PK2 from the SCN is an inhibitory signal for the circulating corticosterone under regular circadian conditions. Lesion studies have implicated that signals from the SCN suppress the corticosterone surge in response to stress. The present invention demonstrates that PK2^{-/-} mice displayed a higher corticosterone level after swimming stress, suggesting PK2 as a suppressor to corticosterone level under stressful condition. The observation that PK2 as a suppressor to corticosterone level under both normal and stressful conditions provides a molecular link between circadian and non-circadian (stressful) regulation of corticosterone level.

It is evident that there exists an intimate link between the circadian timekeeping system and stress regulation as both circadian clock and stress response are expected to affect the neural pathways that control vigilance level, hormones secretion, cardiovascular system and body temperature. The present invention indicates that the same molecule (PK2) regulates the circadian rhythm as well as stress response indicating that these two processes are indeed linked together at the molecular level. As stress response is likely to be central for the pathophysiology of various mood disorders, which are also known to have important circadian components, the manipulations in the PK2/PKR2 system that regulates both stress response and circadian clock may be advantageous for the treatment of these debilitating disorders.

### EXAMPLE II

### Experimental Methods

PK2^{-/-} mice were generated by homologous recombination as described. Male PK2^{-/-} mice and their littermate wild-type mice of C57BL/6×129/Ola hybrid background were used in most of the experiments except sleep/wakefulness recording, in which animals in C57BL/6 background were used. Experimentally naïve mice (age of 3-5 months) were housed in standard cages with food and water available ad libitum unless otherwise indicated. All procedures regarding the care and use of animals are in accordance with institutional guidelines.

Anxiety-like behaviors were measured. The plus maze consisted of two open (30×5 cm) and two wall-enclosed arms (30×5×15 cm) connected by a central platform (5×5 cm). The apparatus was elevated 75 cm above the floor. Behavioral testing was started by placing a mouse in the central area facing a closed arm in which the animal usually enters first. Exploratory behavior was monitored over a period of 5 minutes. Numbers of entries into open arms, time traveled in open and closed arms, general activity, and latency until the first open-arm entry were recorded and quantified. Entries were defined as four paws of an animal entering a new zone.

The light-dark box was divided into a lit compartment (30×20×25 cm, illumination 400 lux) and a dark compartment (15×20×25 cm) connected by a 4-cm tunnel. The experiment was started by placing the animal in the dark compartment.

Numbers of transitions, total time spent in the lit compartment, and latency until the first exit were recorded on video and quantified during 10 minutes.

The Porsolt forced swim test was utilized. Animals were placed individually in cylinders (height 25 cm, diameter 11 cm) filled with 8-cm-deep water (temperature 22±1° C.) for 6 minutes The total period of immobility during the 6 minutes was recorded. Immobility was defined as no volitional body or limb movement.

Pain perception and stress-induced analgesia was measured. All the pain tests were carried out on a hot plate with a 55° C. surface, on which no basal difference was observed between WT and PK2^{-/-} mice. The cut-off time was set to 1 min to prevent tissue damage. Stress was induced by forced swimming for 3 min in 10° C. water, or suspending the tail for 5 minutes.

Core Body temperature and locomotion was measured. A radio transmitter device (G2 E-mitter; Mini-Mitter, Sunriver, Oreg.) used to measure body temperature and locomotor activity was implanted in the abdominal cavity by sterile technique under general anesthesia. Body temperature and locomotor activity were recorded by a receiver board (ER-4000 energizer receiver, Mini-Mitter, Sunriver, Oreg.) underneath the cage and were stored in a personal computer every 5 minutes The mice were allowed to recover for at least 2 weeks before the experiments. The ambient temperature was 21° C. The cage change was done at ZT 4 by transferring the mouse into a new cage with food and water; and the body temperature and locomotion was monitored for another 2 hours.

Food intake was measured. Mice food intake measurement was performed using a Comprehensive Lab Animal Monitoring System (CLAMS, Columbus Instruments, Columbus, Ohio). Feeding data were collected every 10 min for each mouse over a period of 5 days. The daily food intake was the total food consumed during 24 hours. For rats, the food pellets in the food bin were measured manually every 4-8 hours after drug infusion. The food intake was the total amount of food loaded subtracted by the amount of food left.

Recording and analysis of sleep/wake was performed. The sleep/wake recording in rats was carried out as described in reference. Digital recording was visually scored as slow wave sleep (SWS), rapid eye movement sleep (REM) and active wake (AW). For the mice, electroencephalogram (EEG) and electromyogram (EMG) signals were recorded using a lightweight implant and cabling procedure as described. After surgery, mice were allowed to recover for at least 7 days and allowed to adapt for at least 7 days after the connection of a lightweight cable. EEG/EMG signals were collected using a Grass model 15A94 Quad AC amplifier and digitized at a sampling rate of 256 Hz. The signals were digitally filtered (EEG, 0.3-30 Hz; EMG, 2-100 Hz) and semi-automatically scored in 4 second epochs as wake, NREM, or REM sleep using SleepSign sleep scoring software (Kissei Comtec America, California). The total sleep is the sum of NREM and REM sleep. The preliminary scoring was visually inspected and corrected when appropriate.

Intracerebroventricular infusion was utilized. Rats with cannula fit to the lateral ventricle were purchased from Charles River laboratory. For mice, a 26-gauge stainless steel indwelling cannula (Plastics One, Roanoke, Va.) was implanted 2.6 mm below the skull surface into the lateral ventricle (1.1 mm lateral to bregma) as described previously. A dummy cannula was inserted to maintain potency. Injections were performed using a 33-gauge stainless steel injector attached to PE-10 tubing fitted to a 10 μl Hamilton syringe. Injections began 7-10 days after mice recovered from surgery.

Determination of corticosterone level was performed. Wild-type and PK2^{-/-} mice were housed individually before collection of blood samples. For the basal levels, blood samples were collected by retro-orbital eye bleeding within 45 s of initial disturbance of the cages at ZT 1. Blood samples also were collected immediately, or 90 min after stress (3-min cold-water swimming). Plasma corticosterone levels were determined using radioimmunology assay kit (ICN Biomedicals, Costa Mesa, Calif.) according to the instructions.

In situ hybridization was performed for detection. Animals were killed and brains were quickly dissected and stored at −80° C. until use. In situ hybridization was performed on coronal sections as described previously. The ³⁵S-hybridized slices were then coated with Kodak NTB2 autoradiographic emulsion and exposed for 1 month at 4° C.

Statistical analyses was performed. A repeated-measures ANOVA followed by unpaired t test was used to analyze the data for differences between genotypes or treatments. All statistical analysis was performed using Prism 4.4 (GraphPad Software, San Diego, Calif.).

### EXAMPLE III

### Intracerebroventricular (ICV) Infusion of PK2 Enhanced the Behavioral Stress Responses

To investigate the possible role of PK2 in behavioral stress responses, the inventors systemically examined the effects of PK2 delivered by ICV. As shown in FIG. 2A, PK2 significantly increased arousal level when infused at zeitgeber time (ZT). A similar effect on arousal increase when PK2 was infused at ZT 2 was also observed. Similar circadian phase-independent suppression of food intake by PK2 was also observed (FIG. 2B). Inventors further investigated the effect of PK2 infusion on the anxiety-like level in mice. As shown in FIG. 2C, PK2 infusion led to significantly less time spent in the lit compartment in a light-dark box assay, without significant effect on general activity, as reflected on the total entries. The light-dark box utilized the natural aversion of rodents to the bright light and open field; usually, the animals with elevated anxiety will spend less time in the lit compartment. Moreover, PK2 injection significantly increased the time spent on immobility in a forced swimming test (FIG. 2D), a sign of depression-like behavior. Taken together, these studies demonstrated that PK2 infusion facilitates the stress responses in animals.

### EXAMPLE IV

### Impaired Response of PK2^{-/-} Mice to New Environments

This example illustrates the differential levels of arousal period between wild type and PK2^{-/-} mice in response to environmental disturbances.

Applicants have previously shown that PK2^{-/-} mice showed a significantly shorter arousal period after cage change than wild-type controls, indicating an impaired response to environmental disturbance in PK2^{-/-} mice. A similar altered response was observed while monitoring the metabolic parameters with a Comprehensive Laboratory Animal Monitor System (CLAMS, Columbus, Ohio). As shown in FIG. 3A, the food intake in wild-type mice was significantly lower in day 1 than in day 3-4. This phenomenon has been interpreted as exposing to external feeding chambers in CLAMS system suppresses food intake. However, PK2 mice did not exhibit such apparent food intake difference between day 1 and day 3-4.

The core body temperature and locomotor activity was then observed in wild-type and PK2^{-/-} mice in response to a cage change at ZT (during a rest period for mice). As shown in FIGS. 3B and 3C, the cage change increased body temperature and locomotor activity in both wild-type and PK2^{-/-} mice initially. However, wild-type mice exhibited a significant hyperactivity and elevated core body temperature as compared to PK2 mice. During the period of 45 to 90 min after cage change, the body temperature of PK2 mice was significantly lower than that of wild-type mice. Similarly, the locomotor activity of PK2^{-/-} mice during 40 to 75 min after cage change was significantly smaller than that of wild-type mice. Taken together, these results consistently demonstrated that PK2^{-/-} mice showed impaired responses to new environments in terms of activity, arousal, body temperature and food intake.

### EXAMPLE V

### Reduced anxiety-Like Behaviors in PK2^{-/-} Mice

This example illustrates the differential levels of anxiety-like behaviors displayed in PK2^{-/-} mice.

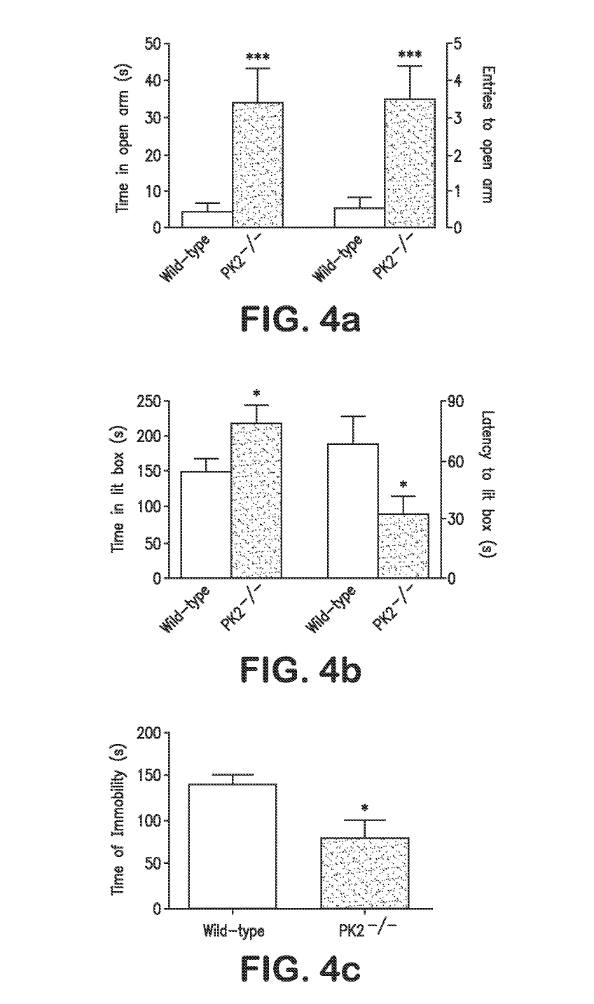

The impaired response of PK2^{-/-} mice to new environments implied the reduction of anxiety-like level in these mutant mice. The anxiety-like behaviors with two standard paradigms was then quantified; elevated plus maze and light-dark box. In the elevated plus maze assay, PK2^{-/-} mice spent significantly more time in the open arm (FIG. 4A). Moreover, the total entries into open arm were significantly higher in PK2^{-/-} mice than wild-type mice (FIG. 4A), while the dips to the open arms were indistinguishable between genotypes. Consistent with the observation from elevated plus maze, PK2^{-/-} mice spent significantly more time in the lit compartment when tested in a light-dark box paradigm (FIG. 4B). PK2^{-/-} mice also showed significant shorter latency to enter the lit box (FIG. 4B), while the latency to the dark box was indistinguishable between genotypes. Taken together, these results indicated the PK2^{-/-} mice displayed reduced anxiety-like behaviors.

### EXAMPLE VI

### PK2^{-/-} Mice Showed Antidepression-Like Behavior

Depression is an important symptom accompanied with chronic stress and anxiety. The depression-like behavior with a forced swimming test was observed. In this swimming test, animals were forced to swim (21° C.) for 6 minutes. The animals tried to escape at early phase, and then they adopted an energy-saving strategy (i.e., immobility) after initial unsuccessful escape. As shown in FIG. 4C, PK2^{-/-} mice spent significantly less time on immobility than wild-type mice during this 6-min forced swimming test. Thus, PK2^{-/-} mice displayed impaired response to forced swimming.

### EXAMPLE VII

### Absence of Fasting-Induced Arousal Increase in PK2^{-/-} Mice

Animals respond to reduced food availability by becoming more wakeful and active, with phases of increased arousal and locomotor activity supporting food seeking activity. To examine whether PK2 signaling is required for adaptive arousal responses to a metabolic stress of starvation, states of wakefulness, non-rapid eye movement (NREM) sleep, and rapid eye movement (REM) sleep during food deprivation were recorded. After baseline (fed ad lib) recording under regular light-dark cycles, animals were fasted for 36 hours beginning with onset of the dark phase (FIGS. 5A and 5C). Analysis of sleep/wake states revealed significant increases of arousal in wild-type mice during fasting; particularly, there was a robust increase in arousal after 18 hours of fasting (FIGS. 5A and 5B). In contrast, PK2^{-/-} mice exhibited no appreciable increase in arousal during the entire fasting period (FIGS. 5C and 5D). Consistent with impaired response to fasting in the wake/sleep recording, PK2^{-/-} mice lost significantly less body weight during 48 hours of fasting than wild-type controls.

### EXAMPLE VIII

### PK2^{-/-} Mice Showed Impaired Stress Induced Antinociception (SIA)

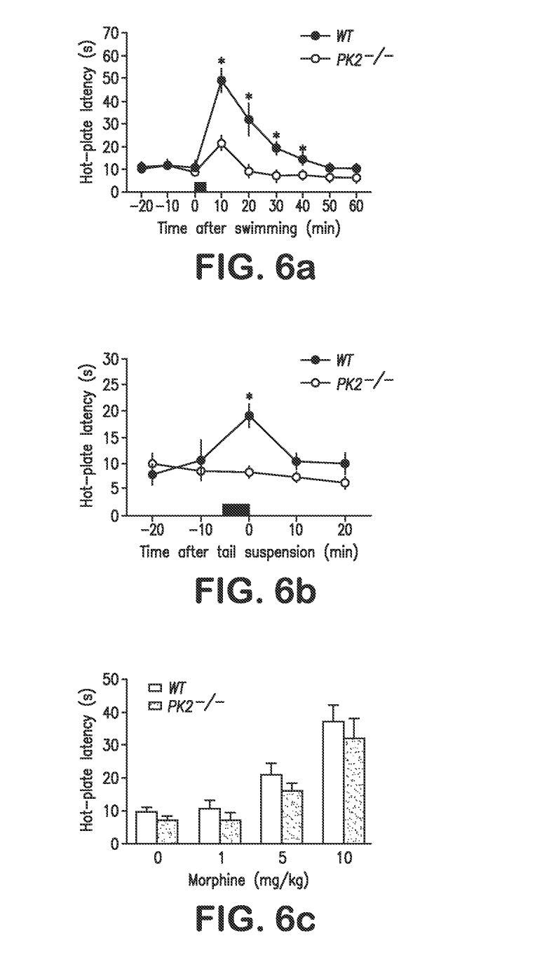

It is well known that a variety of stressors induce antinociception, a phenomenon termed SIA. SIA is thought to benefit the subjects to cope with stressful situations without being bothered by the pain. The SIA in PK2^{-/-} mice and wild-type controls with cold-water swimming (10° C.) were measured, which is known to induce analgesia in a largely opiate-independent way. As shown in FIG. 6A, wild-type mice showed significant SIA after 3 minutes of swimming at 10° C. It should be noted that most of the wild-type mice reached the hot-plate cut-off time (1 min) on the 55° C. surface at 7 minutes after swimming. Additionally, the SIA lasted for almost 40 min in wild-type mice. In contrast, SIA was significantly attenuated in PK2^{-/-} mice, with only significant threshold increase at 7 min after swimming (FIG. 6A). To rule out the possibility that alterations in the processing of peripheral temperature could contribute to the defect in PK2^{-/-} mice, SIA induced by a temperature-independent stress was also measured, such as tail suspension. Tail suspension was conducted by suspending mice with the tail for a short period (5 min) As shown in FIG. 6B, tail suspension can induce slight but significant analgesia in wild-type mice. However, such SIA was absent in PK2^{-/-} mice. In contrast to cold swimming-induced analgesia, the analgesia induced by tail suspension was opiate-dependent. These data indicated that PK2 is essential for both opiate-dependent and independent SIA. It is important to note that morphine-induced analgesia was indistinguishable between WT and PK2^{-/-} mice (FIG. 6C).

### EXAMPLE IX

### Stress Induced Corticosterone Secretion in PK2^{-/-} Mice

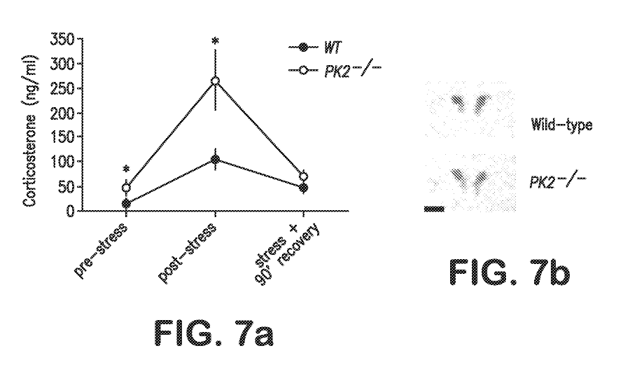

The activation of HPA axis, which can be estimated by the elevated plasma corticosterone levels, is a critical integrator of stress response. Moreover, circulating glucocorticoid plays important roles in the feedback regulation of stress response. To determine if PK2^{-/-} mice have deficit in the HPA axis activation, the circulating corticosterone levels in PK2^{-/-} and wild-type mice before was measured, immediately and 90-min after 3-min of cold-water swimming. As shown in FIG. 7A, PK2^{-/-} mice showed significantly higher basal corticosterone levels at ZT1, which was due to the attenuated circadian rhythms in PK2^{-/-} mice. Three minutes of cold-water swimming led to a striking increase of the corticosterone level in both wild-type and PK2^{-/-} mice. In fact, PK2^{-/-} mice displayed even greater increase of corticosterone level in response to cold-water swimming, suggesting that the stress can be perceived by PK2^{-/-} mice and this stress elicited the activation of HPA axis in these mice. At 90 min after stress, the corticosterone level in both genotypes returned to basal level. Furthermore, no genotypic difference of CRF expression was observed in the PVN (FIG. 7B). These results indicate HPA activation by stress is intact in PK2^{-/-} mice.

Throughout this application various publications have been referenced within parentheses. The disclosures of these publications in their entireties are hereby incorporated by reference in this application in order to more fully describe the state of the art to which this invention pertains.

Although the invention has been described with reference to the above example, it will be understood that modifications and variations are encompassed within the spirit and scope of the invention. Accordingly, the invention is limited only by the following claims.

What is claimed is:

1. A method of modulating behavioral stress response or anxiety-like behaviors in a subject in need thereof comprising administering to the subject an effective amount of an agent that binds to prokineticin 2 (PK2) receptor, thereby modulating behavioral stress response or anxiety-like behaviors in the subject.

2. The method of claim 1, wherein the subject presents symptoms of behavioral stress response or anxiety-related behaviors.

3. The method of claim 2, wherein the agent decreases behavioral stress response or anxiety-related behaviors.

4. A method for identifying an agent that modulates behavioral stress response or anxiety-like behaviors in a subject, comprising:
a) contacting a sample comprising a PK2 receptor with at least one test agent, under conditions suitable for the PK2 receptor to bind to the test agent, wherein binding of the PK2 receptor to the test agent promotes a predetermined signal; and
b) comparing the predetermined signal to a control signal, wherein an altered signal is indicative of an agent that modulates behavioral stress response or anxiety-like behaviors.

5. The method of claim 4, wherein an increase in the predetermined signal as compared to the control signal is indicative of an agent that decreases behavioral stress response or anxiety-like behaviors.

6. The method of claim 4, wherein a decrease in the predetermined signal as compared to the control signal is indicative of an agent that increases behavioral stress response or anxiety-like behaviors.

7. The method of claim 4, wherein the test agent is administered to a subject to modulate behavioral stress response and anxiety-like behaviors.

8. The method of claim 4, wherein the sample is contacted with two or more agents.

9. The method of claim 4, wherein the PK2 receptor is a mouse PK2 receptor.

10. The method of claim 4, wherein the PK2 receptor is a primate PK2 receptor.

11. The method of claim 4, wherein the control signal is generated by binding of human PK2 to PK2 receptor.

12. The method of claim 2, wherein the subject is a mammal.

13. The method of claim 4, wherein the subject is selected from human, non-human primate, rat and mouse.

14. The method of claim 4, further comprising detecting behavioral stress response or anxiety-like behaviors in the subject.

15. The method of claim 12, wherein the behavioral stress response and anxiety-like behaviors are detected by EEG and EMG waves.
